(19) United States
(12) Patent Application Publication
(10) Pub. No.: US 2013/0338560 A1
(43) Pub. Date: Dec. 19, 2013

(54) METHOD AND APPARATUS FOR DETERMINING ACCESS FLOW

(71) Applicant: GAMBRO LUNDIA AB, (US)

(72) Inventors: Bernard Bene, Irigny (FR); Nicolas Goux, Craponne (FR); Per Hansson, Linhamn (SE); Thomas Hertz, Lund (SE); Olof Jansson, Vellinge (SE); Roland Persson, Limhamn (SE); Jan Sternby, Lund (SE); Perry Asbrink, Malmo (SE)

(73) Assignee: GAMBRO LUNDIA AB, Lund (SE)

(21) Appl. No.: 13/872,728

(22) Filed: Apr. 29, 2013

Related U.S. Application Data
(63) Continuation of application No. 10/503,766, filed on Aug. 6, 2004, now abandoned.

(30) Foreign Application Priority Data
Feb. 8, 2002 (SE) 0200370.5

(51) Int. Cl. A61M 1/30

(52) U.S. Cl. CPC A61M 1/30
USPC 604/6.09

(57) ABSTRACT

A method and apparatus for determining a fluid flow rate in a blood access having an upstream position and a downstream position using a dialysis system. The dialysis system includes a dialyzer having a semi permeable membrane delimiting a first chamber through which blood removed from said blood access passes, and a second chamber through which dialysis liquid passes. In addition, an arterial line and a venous line are connected to an inlet and an outlet of the first chamber, respectively.

### RELATED APPLICATIONS

This application is a continuation application of U.S. application Ser. No. 10/503,766 filed Aug. 6, 2004 and which is national phase of International Patent Application No. PCT/IB02/04537 and claims the priority of Swedish Patent Application No. 0200370-5, filed on Feb. 8, 2002, the contents of which are incorporated herein by reference.

### TECHNICAL FIELD

The present invention relates to a method and apparatus for determining fluid flow rate in a patient's blood access. More particularly, the invention relates to the calculation of the fluid flow rate in the blood access based on conductivity measurements of the post dialyzer or other blood treatment unit effluent fluid.

### BACKGROUND

There are several types of treatments in which blood is taken out in an extracorporeal blood circuit. Such treatments involve, for example, hemodialysis, hemofiltration, hemodiafiltration, plasmapheresis, blood component separation, blood oxygenation, etc. Normally, blood is removed from a blood vessel at a blood access and returned to the same blood vessel.

In hemodialysis and similar treatments, a blood access commonly surgically created in the nature of a arterio-venous shunt, commonly referred to as a fistula. Blood needles are inserted in the fistula. Blood is taken out from the fistula via a needle at an upstream position and blood is returned to the fistula via needle at a downstream position.

The arterio-venous shunt or fistula is blood access having capability of providing a high blood flow and being operative during several years and even tens of years. It is produced by operatively connecting, for example, the radial artery to the cephalic vein at the level of the forearm. The venous limb of the fistula thickens during the course of several months, permitting repeated insertion of dialysis needles.

An alternative blood access to the fistula is the arterio-venous graft, in which a connection is generated from, for example, the radial artery at the wrist to the basilic vein. The connection is made with a tube graft made from e.g. autogenous saphenous vein or from polytetrafluorethylene (PTFE, Teflon). The needles are inserted in the graft.

A further example of a blood access is a silicon, dual-lumen catheter surgically implanted into one of the large veins.

Further type of blood access find use in specific situations, like a no-needle arterio-venous graft consisting of a T-tube linked to a standard PTFE graft. The T-tube is implanted in the skin. Vascular access is obtained either by unscrewing a plastic plug or by puncturing a septum of said T-tube with a needle. Other methods and devices are also known.

During the above blood treatment therapies, hemodialysis for instance, it is desirable to obtain a constant blood flow rate of 150-500 ml/min or even higher, and the access site must be prepared for delivering such flow rates. The blood flow in an AV fistula is often 800 ml/min or larger, permitting delivery of a blood flow rate in the desired range.

In the absence of a sufficient forward blood flow, the extracorporeal circuit blood pump will take up some of the already treated blood entering the fistula via the venous needle, so called access or fistula recirculation, leading to poor treatment results and progressive reduction of treatment efficiency.

A common cause of poor flow with AV fistulas is partial obstruction of the venous limb due to fibrosis secondary to multiple venipunctures. Moreover, stenosis causes a reduction of access flow.

It has been found that access flow rate often exhibit a long plateau time period with sufficient access flow, followed by a short period of a few weeks with markedly reduced access flow leading to recirculation and ultimately access failure. By constantly monitoring the evolution of the access flow during consecutive treatment sessions, it is possible to detect imminent access flow problems. Proper detection of access flow reduction may help in carrying out a maintenance procedure on the access thereby avoiding any access failure.

A non-invasive technique that allows measurement of flow through AV fistulas and grafts is colour Doppler ultrasound. Magnetic Resonance Imaging (MRI) has also been used. However, these techniques require expensive equipment and are not easily used in the dialysis clinic environment.

Several methods have been suggested for monitoring recirculation and access flow. Many of these methods involve injection of a marker substance in blood, and the resultant recirculation is detected. The methods normally involve measurement of a property in the extracorporeal blood circuit. Examples of such methods can be found in U.S. Pat. No. 5,685,989, U.S. Pat. No. 5,595,182, U.S. Pat. No. 5,453,576, U.S. Pat. No. 5,510,716, U.S. Pat. No. 5,510,717, U.S. Pat. No. 5,312,550, etc.

Such methods have the disadvantage that they require the injection of the marker substance and external equipment for the measurements.

More recently, EP 928 614 and WO 00/24440, suggest to measure a post dialyzer concentration of a substance, in particular urea in the effluent fluid before and after a flow reversal, i.e. before the flow reversal the arterial line carries blood from an upstream position of the blood access, and the venous line carries blood towards a downstream position of the blood access, whereas the arterial line carries blood from an downstream position of the blood access, and the venous line carries blood towards a upstream position of the blood access after the flow reversal. A valve for such reversal is shown in i.e. U.S. Pat. No. 5,605,630 and U.S. Pat. No. 5,894,011. A disadvantage in these methods is the requirement for special equipment for measuring the urea concentration. Urea sensors are as such available but they are not standard equipment for most of the dialysis monitors and they have also a considerable maintenance costs.

### SUMMARY

On this background, it is the object of the present invention to provide a method of the kind referred to initially, which is less expensive, easier to implement and easier to operate. This object is achieved creating a concentration difference between the blood and the dialysis liquid, and measuring the post dialyzer concentration or conductivity before and after a flow reversal. The creation of a difference in concentration for the purpose of measuring the fluid flow in the blood access allows for a significant increase in precision of the measurement.

It is another object of the invention to provide a blood treatment apparatus, a dialysis apparatus for instance, of the kind referred to above, able to measure blood access flow, less expensive and easier to operate than the known apparatuses.

By providing means for creating a difference in conductivity between the dialysis fluid and blood and by providing a post treatment unity conductivity cell, the apparatus can determine the blood access flow, with relatively inexpensive modifications to conventional dialysis apparatuses.

According to a preferred embodiment, a first and second concentration or conductivity are measured on the post treatment unit fluid flowing downstream the treatment unit, or so called effluent fluid.

During normal dialysis a blood flow in a first direction is created by operating a blood pump, in which the arterial line carries blood from said upstream position of said blood access, and the venous line carries blood towards said downstream position of said blood access (normal configuration of the lines).

A blood flow in a second direction, in which said arterial line carries blood from said downstream position of said blood access, and said venous line carries blood towards said upstream portion of said blood access (reversed configuration of the lines), may be created by
manually connecting the arterial line to the downstream position of the blood access and the venous line to an upstream position of the blood access, or by
connecting the arterial line to both the upstream and the downstream position of the blood access and connecting the venous line to both the upstream and the downstream position of the blood access, closing one of the connections between the arterial line with the blood access and opening the other and closing one of the connections between the venous line with the blood access and opening the other, or by
providing a valve able to connect the arterial line with the upstream position of the access point and the venous line with the downstream position of the access point in a first position of said valve and able to connect the arterial line with the downstream position of the access point and the venous line with the upstream position of the access point in a second position of said valve.

The calculation of the fluid flow rate in the blood access is carried out by using the formula:

Qa=f(Cr,Ci,Cn,Quf,Tr);

According to an embodiment the following formula can be used:

Qa=(Tr−Quf)*(Cr−Ci)/(Cn−Cr),

In which Qa is the fluid flow rate in the blood access, Tr transport rate of substances over the semi permeable membrane of the treatment unit referred to the venous and arterial lines in normal condition, Quf is the ultrafiltration flow rate, Cr is the post treatment unit conductivity after flow reversal, Ci is pre treatment unit conductivity, and Cn is the post treatment unit conductivity before the flow reversal.

For determination of the transport rate Tr, the effective ionic dialysance D can be used. The effective ionic dialysance D determined for example as described in EP 658 352. Alternatively, the transport rate can be derived from experience values of a particular dialyzer.

The effective urea clearance, determined by other methods known in the art, can also be used for the transport rate Tr, since it has been found to be very similar to effective ionic dialysance.

According to an embodiment of the invention the method (and corresponding blood treatment apparatus) for determining Qa comprises the following steps:
a. circulating a first dialysis liquid into the second chamber inlet of said treatment unit, said first dialysis liquid presenting a treatment concentration for one or more substances, then
b. increasing or decreasing at a time Ti the concentration of the substance in the dialysis liquid for circulating to the second chamber inlet, during a time interval T, a second liquid having a concentration Ci for said one or more substances different from the concentration of the same substances in blood,
c. switching the venous and arterial lines between one to the other of said normal and reversed configurations during the time interval T,
d. obtaining the first post treatment unit conductivity of the dialysis liquid or first concentration of said substance in the dialysis liquid, relating to the dialysis liquid before switching the venous and arterial lines and during said time interval T,
e. obtaining the second post treatment unit conductivity of the dialysis liquid or second post treatment unit concentration of said substance in the dialysis liquid, relating to the dialysis liquid after switching of the venous and arterial lines and during said time interval T, during said time interval T, the concentration Ci of said substance(s) being kept substantially constant.

According to another feature of the invention it may be provided to that, during said time interval T, the following consecutive sub-steps are executed:
a. First configuring the said arterial and venous lines according to the normal configuration for obtaining said first concentration or first conductivity, and then
b. configuring the arterial and venous lines according to the reversed configuration for obtaining said second concentration or conductivity.

Alternatively, during said time interval T, the following consecutive sub-steps may be provided with:
a. First, configuring the said arterial and venous lines according to the reversed configuration for obtaining said first post treatment unit concentration or conductivity, and then
b. configuring the arterial and venous lines according to the normal configuration for obtaining said second concentration or conductivity.

Thanks to this alternative option it is possible to first configure the lines in the reversed configuration for the execution of the Qa determination. As for Qa calculation, a measurement in the normal configuration is also necessary, by starting in reversed configuration and then passing to normal configuration there is no risk to leave the lines in reverse configuration which would lead to a reduced treatment efficiency.

Another advantage with this modified procedure is that we have an automatic indication that the lines have actually been returned to normal for the rest of the treatment, otherwise there will be no access flow measurement. With the original procedure it is much more difficult for the machine to detect if the lines are left in the reversed position for the rest of the treatment.

In term of fistula flow determination, notice that two things will happen if we go from reversed lines back to normal instead of the other way around. First of all, the clearance measured at the conductivity change will be a clearance with reversed lines. This clearance is lower than the normal clearance, how much is determined by the access flow rate. Secondly, the conductivity change caused by returning the lines to normal will go in the opposite direction to normal. The sign of the conductivity change can be handled just by using the absolute value of the change, but the lower clearance value needs to be handled by a change in the formula. As the access flow rate (A) depends on normal configuration clearance (Kₙ), ultrafiltration rate (UF) and reversed flow configuration clearance (Kᵣ) according to

$$A = ( {K}_{n} - UF ) \cdot ( \frac{{K}_{r}}{{K}_{n} - {K}_{r}} ) \qquad ( 1 )$$

then

A=(Kₙ−UF)·R (2)

with R determined from the inlet conductivity (Cᵢ), and the outlet conductivities in normal (Cₙ) and reversed (Cᵣ) positions according to

$$R = ( \frac{{c}_{r} - {c}_{i}}{{c}_{n} - {c}_{r}} ) \qquad ( 3 )$$

Combining (1) and (2) we see that

Kₙ·R=Kᵣ·R+Kᵣ (4)

Access flow rate can therefore be calculated as

A=(Kₙ−UF)·R=Kᵣ·R+Kᵣ−UF·R=(Kᵣ−UF)·R+Kᵣ (5)

Since Kᵣ is the measured clearance when the lines are reversed, the only modification to the formula for access flow that has to be made if the lines are reversed from the beginning is that we must add the measured clearance. Note however for the calculation of R that Cₙ and Cᵣ will switch positions time wise if the lines are reversed from the start (i.e. Cᵣ will be measured before Cₙ).

Note that in the present description and in the claims Cₙ refers always to conductivity-concentration of the effluent dialysis fluid in normal configurations of the lines while Cᵣ refers always to conductivity-concentration of the effluent dialysis fluid in reversed configuration of the lines. If the time sequence adopted is first reversed than normal configuration: the first post treatment unit conductivity-concentration of the dialysis liquid is Cᵣ while the second post treatment unit conductivity-concentration is Cₙ. If the time sequence adopted is first normal than reversed configuration: the first post treatment unit conductivity-concentration of the dialysis liquid is Cₙ while the second post treatment unit conductivity-concentration is Cᵣ.

During execution of the above-disclosed method, the post treatment unit conductivities (first and second) are measured after a delay allowing equilibrium to establish.

According to a feature of the invention, the post treatment unit conductivity after the flow reversal is measured at various intervals or continuously so that the value of the conductivity at the time of the flow reversal can be determined by extrapolating the measured values backwards to the moment of the flow reversal. In this way the method can compensate for drift of parameters between the time when the flow is reversed until the time where a substantial equilibrium is reached.

### BRIEF DESCRIPTION OF THE DRAWINGS

In the following detailed portion of the present description, the invention will be explained in more detail with reference to the exemplary embodiments shown in the drawings, in which

FIG. 1 is a partially schematic view of a forearm of a patient provided with an AV fistula.

FIG. 2 is a schematic diagram of an extracorporeal circuit and part of the fluid path of a dialysis machine.

FIG. 3 is a schematic diagram of an extracorporeal circuit including a flow reversal valve.

FIG. 4 is the schematic diagram of FIG. 3, with the valve turned for reversed blood flow

FIG. 5 is a graph showing the conductivities before and after flow reversal, and

FIG. 6 is another graph showing the conductivities before and after flow reversal.

### DESCRIPTION OF ILLUSTRATIVE EMBODIMENTS

For the purpose of this description, a blood access is a site in which a fluid in a tube can be accessed and removed from and/or returned to the tube. The tube may be a blood vessel of a mammal, or any other tube in which a fluid is flowing. The general term blood access as used here includes arterio-venous fistulas, arterio-venous grafts, and dual-lumen catheters amongst other similar types of blood access that allow for an upstream access position and a downstream access position.

The general terms dialyzer or blood treatment unit as used here include filters for hemodialysis, hemofilters, hemodiafilters, plasmafilters and ultrafilters.

The fluid flow rate is the flow rate of the fluid in the tube or blood vessel immediately upstream of the blood access, denoted Qa.

The general term dialysis as used here includes hemodialysis, hemofiltration, hemodiafiltration and therapeutic plasma exchange (TPE), among other similar treatment procedures.

The general term effluent fluid as used here refers to the dialysis fluid downstream of the dialyzer or blood treatment unit.

The general term “transport of substances or ions though the semi permeable membrane” includes any parameter that is indicative of the rate at which substances or ions pass through the dialyzer membrane. Examples of such parameters are, clearance, urea clearance, dialysance, ionic dialysance and effective ionic dialysance.

The general term ionic dialysance as used here refers to a variable that expresses the transport of ions through the dialyzer membrane. The ionic dialysance is ion dependent, i.e. different ions have different dialysance values. It is also dependent on blood flow, dialysate flow and Quf, so during measurements when determining the access flow these must preferably be held constant. The effective ionic dialysance, herein denoted D, further depends on recirculation effects in the fistula and the cardiopulmonary circuit, and is obtained for example as described by EP 658 352. The major ions determining the conductivity of dialysate liquid are sodium and chloride

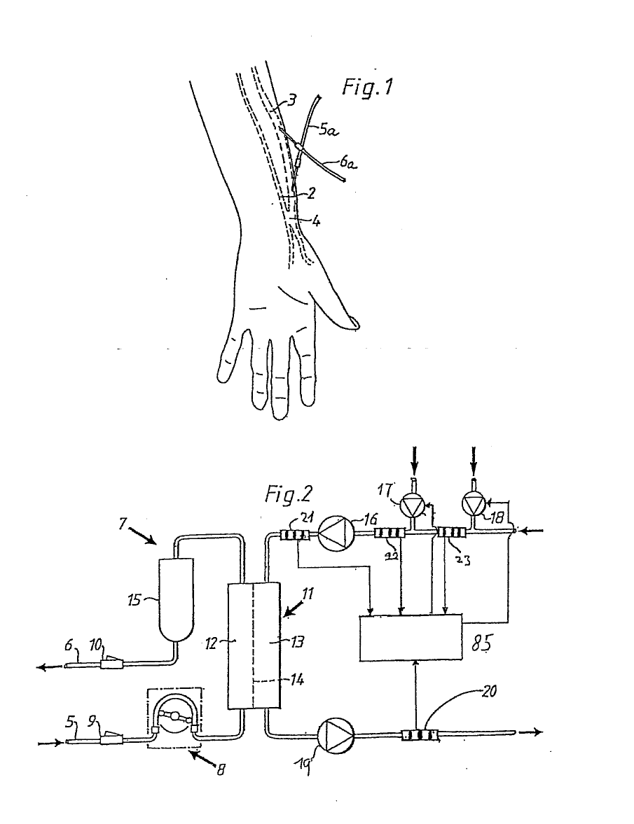

FIG. 1 discloses a forearm 1 of a human patient. The forearm 1 comprises an artery 2, in this case the radial artery, and a vein 3, in this case the cephalic vein. Openings are surgically created in the artery 2 and the vein 3 and the openings are connected to form a fistula 4, in which the arterial blood flow is cross-circuited to the vein. Due to the fistula, the blood flow through the artery and vein is increased and the vein forms a thickened area downstream of the connecting openings. When the fistula has matured after a few months, the vein is thicker and may be punctured repeatedly. Normally, the thickened vein area is called a fistula.

An arterial needle 5a, to which is connected a piece of tube, is placed in an upstream position in the fistula, in the enlarged vein close to the connected openings and a venous needle 6a, to which is connected a piece of tube, is placed in a position downstream of the arterial needle, normally at least five centimeters downstream thereof.

As described above, the blood access can also be an arterio-venous graft, a double lumen catheter or other similar arrangements.

The needles 5a and 6a are connected to a tube system, shown in FIG. 2, forming an extracorporeal circuit 7 comprising a blood pump 8, such as a peristaltic pump. The blood pump propels blood from the fistula, through the arterial needle, the extracorporeal circuit, the venous needle, and back into the fistula.

The extracorporeal blood circuit 7 shown in FIG. 2 further comprises an arterial clamp 9 and a venous clamp 10 for isolating the patient from the extracorporeal circuit should an error occur.

Downstream of pump 8 is a dialyzer 11, comprising a first, so called blood chamber 12 and a second, so called dialysis fluid chamber 13 separated by a semi permeable membrane 14. Further downstream of the dialyzer is a drip chamber 15, separating air from the blood therein.

The bloodline upstream of the dialyzer 11 is referred to as the arterial line 5, whereas the bloodline downstream from the dialyzer 11 is known as the venous line 6. The arterial and venous lines 5 and 6 are able to be configured according to at least a normal configuration, in which said arterial line carries blood from said upstream position of said blood access and said venous line carries blood towards said downstream position of said blood access, and to at least a reversed configuration, in which said arterial line carries blood from said downstream position of said blood access and said venous line carries blood towards said upstream portion of said blood access.

In the normal configuration, blood passes from the arterial needle past the arterial clamp 9 to the blood pump 8. The blood pump drives the blood through the dialyzer 11 and further via the drip chamber 15 and past the venous clamp 10 back to the patient via the venous needle. The drip chamber may comprise an air detector, adapted to trigger an alarm should the blood emitted from the drip chamber comprise air or air bubbles. The blood circuit may comprise further components, such as pressure sensors etc.

The dialysis fluid chamber 14 of the dialyzer 11 is provided with dialysis fluid via a first pump 16, which obtains dialysis fluid from a source of pure water, normally RO-water, mixed with one or several concentrates of ions, varying means including metering pumps 17 and 18 being shown for metering such concentrates. Sensors comprising a conductivity cell 22 and a conductivity cell 23 are provided downstream of the points where the concentrates are mixed into the main fluid steam. The signal of the respective conductivity cell 22,23 is in a closed loop manner compared with the desired conductivity and the speed of the pumps 17 and 18 are controlled in response. A further conductivity cell 21, connected to the protective system of the dialysis machine, is provided downstream from all concentrate mixing steps measuring the final total conductivity. The protective system compares the measured final conductivity with a calculated final conductivity and puts the dialysis machine in a safe state, if anything should have gone wrong in the mixing steps.

A control unit 85 operates said varying means for circulating a dialysis liquid in the second chamber of said treatment unit in such a way that, at least for a time interval T, said dialysis liquid upstream the treatment unit has a concentration (Ci) of one or more substances different from the concentration of the same substance(s) in blood.

According to an embodiment of the invention the difference in concentration is measured as a difference in the conductivity, because most of the components in the dialysis liquid are electrolytes and thus a change in their concentration will inherently lead to a change in the conductivity of the dialysis liquid. It will be understood though, that the invention can also be carried out using the concentration of substances that have no or little effect on the conductivity of the liquid that they are dissolved in, such as urea or glucose.

A preferable range for the dialysate conductivity during the blood access flow measurement is 14.5 to 17.5 mS/cm, preferably about 15 to 16 mS/cm. Thus a conductivity difference between the blood and the dialysate of about 1 to 2 mS/cm is created.

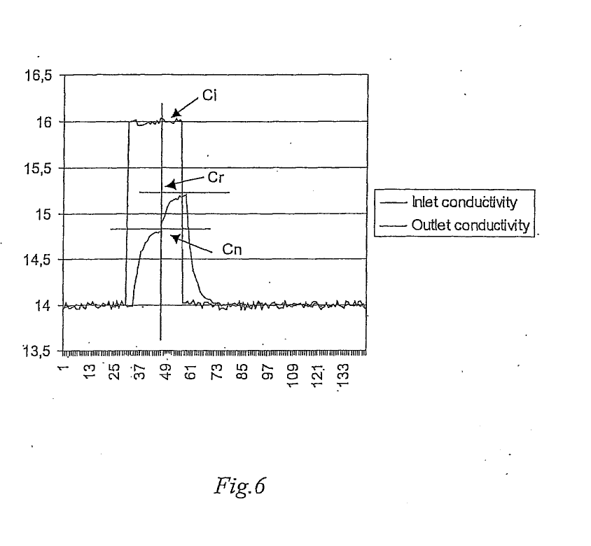

In the specific embodiment shown in FIGS. 5 and 6 an increase in conductivity (concentration of one or more electrolytes) is applied to the fluid upstream the second chamber 13. Said increase starts at time Ti in order to bring the second chamber inlet conductivity to a substantially constant value Ci for a certain time interval T.

According to a first alternative, the invention can work even if instead of an increase a decrease in conductivity or concentration is applied to the fluid at the inlet of the second chamber.

According to a second alternative, if the dialysis liquid inherently has the required difference in conductivity with respect to the blood, then no change in conductivity shall be created for performing the method according to the invention.

A major contribution to the conductivity of the dialysis liquid is sodium chloride. From a physiological standpoint and for best control, the preferred way to adjust the final total conductivity is therefore to change the concentration of sodium chloride. The control unit 85 changes the setting of sodium chloride and in response the speed of metering pump 17 and/or 18 is adjusted as described above. In many types of dialysis apparatus however, the sodium chloride is in a concentrate container together with all the minor amounts of other electrolytes e.g. potassium, magnesium, calcium and peracetic acid, the so called “A concentrate”. This concentrate contributes about 12 mS/cm of the usual final 14 mS/cm conductivity. The remainder of the conductivity comes from the bicarbonate concentrate. In such a dialysis machine (not shown) the conductivity is set by changing the amount of A concentrate in the same way as described above for sodium chloride alone.

Though less attractive from a physiological point of view, it is also possible to change the concentration of all electrolytes, i.e. inclusive bicarbonate simultaneously. It is also possible to change the concentration of any other electrolytes or other components such as glucose.

An exchange of substances between the blood and the dialysis fluid takes place in the dialyzer 11 through the semi permeable membrane 14. The exchange may take place by diffusion under the influence of a concentration gradient, so called hemodialysis, and/or by convection due to a flow of liquid from the blood to the dialysis fluid, so called ultrafiltration.

From the dialysis fluid chamber 14 of the dialyzer is emitted a fluid called the effluent fluid, which is driven by a second pump 19 via a conductivity cell 20 to drain. The conductivity cell measures continuously or at various intervals, the conductivity of the effluent fluid emitted from the dialyzer, to provide an effluent fluid conductivity.

As described above, the present invention provides a method of non-invasively measuring the fluid flow in the fistula immediately before the arterial needle, using the conductivity cell 20 and the dialysis circuit as shown in FIG. 2.

By measuring the first post dialyzer liquid conductivity-concentration during normal dialysis (or normal configuration of the venous and arterial lines) and then reversing the positions of the needles (reversed configuration) and measuring the second post dialyzer conductivity-concentration with the needles in the reversed position, the control unit is able to calculate the blood flow in the blood access, without the addition of any substance to the blood or the dialysis fluid solely for the sake of the measurement.

Note that in order to pass from the normal configuration of the lines to the reversed configuration of the lines the following alternative options can be used.

One way of achieving flow reversal in the needles is by manually disconnecting the needles from the bloodlines and reconnecting the arterial needle to the venous bloodline and the venous needle to the arterial bloodline (not shown). Various other ways for achieving the flow reversal are known to the skilled person.

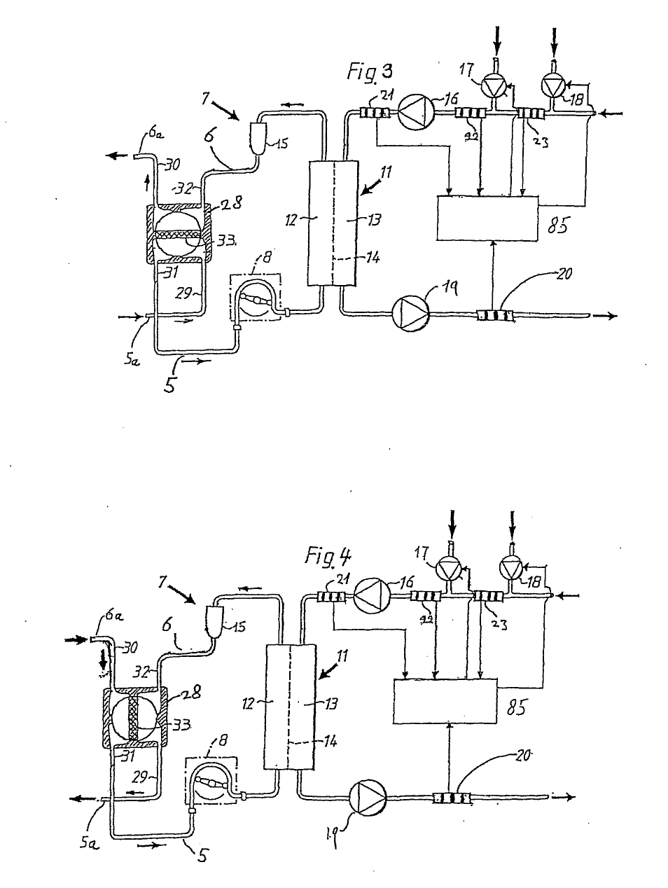

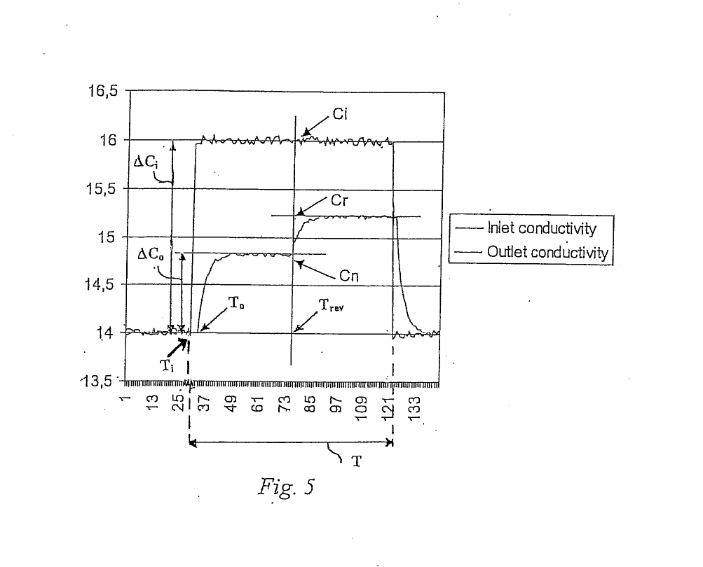

Another embodiment usable for switching the lines between the normal and the reversed condition and vice-versa is shown in FIGS. 3 and 4. These figures relate to a schematic diagram of the dialysis circuit according to FIG. 2 with the addition of a valve 28 to perform the flow reversal. The arterial needle 5a is connected to an arterial inlet line 29 of the valve and the venous needle 6a is connected to a venous inlet line 30 of the valve. The blood pump is connected via arterial line 5 to a first outlet line 31 of the valve and the blood returning from the dialyzer 11 is connected via the venous line 6 to a second outlet line 32 of the valve. The valve 28 comprises a valve housing and a pivotable valve member 33, which is pivotable from the normal position shown on the drawing to a reverse position pivoted 90° in relation to the normal position. In the normal position shown in FIG. 3, the arterial needle 5a is connected to the blood pump 8 and the venous needle 6a is connected to the outlet of the dialyzer, via the drip chamber 15. In the reversed position shown in FIG. 4, the arterial needle 5a is connected to the outlet of the dialyzer and the venous needle 6a is connected to the blood pump 8, as required. Thus the flow is “reversed”, and the arterial line 5 carries blood from a downstream position of the blood access, and the venous line 6 carries blood towards an upstream position of the blood access. According to an embodiment, the dialysis machine automatically controls the change of the valve position.

As mentioned before other systems may be used to pass form a configuration to the other; for instance manually changeable connections in the arterial line to the downstream position of the blood access and in the venous line to an upstream position of the blood access.

Alternatively the lines may be designed to present first conduits connecting the arterial line to both the upstream and the downstream position of the blood access and second conduits connecting the venous line to both the upstream and the downstream position of the blood access. In order to operate the configuration, means for selectively closing one of the first conduits between the arterial line and the blood access and means for selectively closing one of the conduits between the venous line and the blood access can be provided. Such closing means can be manually operable valves or valves controlled by the blood treatment apparatus. Pinch valves, cam valves or clamps having portions active on respective tube portions can be used.

As a further alternative flow distribution means can be used able of connecting the arterial line with the upstream position of the access point and the venous line with the downstream position of the access point, in a first state of said flow distribution means, and able to connect the arterial line with the downstream position of the access point and the venous line with the upstream position of the access point, in a second state of said flow distribution means.

FIGS. 5 and 6 are graphs of measured pre and post dialyzer conductivities. The horizontal axis represent the lapsed times and the vertical axis represent the measured conductivity in mS/cm. In FIGS. 5,6 it is assumed to start with the venous and arterial lines in normal condition and to switch the lines into the reversed condition during the time interval T of change of the conductivity of the dialysis fluid. As already mentioned it is possible to execute the method according to the invention starting with the reversed condition.

For determining the fluid flow rate in the blood access, a gradient between the conductivity of the dialysis fluid (Ci) at the dialyzer inlet and the blood (Cb) is created (FIG. 5). Hereto the conductivity of the dialysis liquid is increased from the conventional value of 14 mS/cm (first dialysis liquid having conductivity which corresponds roughly to the conductivity of blood) to 16 mS/cm (second dialysis liquid). The difference may be of another magnitude and, as already mentioned, can also be created by reducing the conductivity of the dialysis fluid. The conductivity of the second liquid is at least 2 mS/cm (2 milli-Siemens/centimeter) higher than the conductivity of the first liquid if the conductivity of the first liquid is less or equal to 15 mS/cm.

The conductivity gradient is preferably obtained by changing the sodium chloride concentration, but may also be obtained by varying the concentrations of any of the other electrolytes present in dialysis fluid. The change in electrolyte concentration can in advanced dialysis machines such as the Gambro AK 200 S® be executed by changing the settings or programming a step through the user interface. Use of conductivities instead of concentrations is simpler, more reliable, cheaper to implement as it employs the conventional sensors of the treatment apparatus, does not need determination of D or K in two different conditions.

In FIGS. 5 and 6 the conductivity of the dialysis fluid Ci prepared by the dialysis monitor is increased from 14 to 16 mS/cm at time Ti. The conductivity Cn of the post dialyzer fluid, the effluent fluid, will begin to increase at time To with a delay To−Ti caused by the volume of the tubes and the dialyzer. Cn will reach a semi stable value only after some time. Because the increased conductivity of the dialysis liquid causes a transport of ions form the dialysis liquid to the blood, which therefore also slowly increases in conductivity, there will be a slow drift in of the post dialyzer conductivity. The value of Cn may be determined after the respective value has become substantially stable, as shown in FIG. 5. In order to further improve the precision of the method the value of Cn may be extrapolated forward to the point in time of the flow reversal Tᵣₑᵥ. Alternatively, the value of Cn may be determined while it is still increasing by estimating which substantially stable value Cn would have reached after an equilibrium has been established by using numerical methods such as curve fitting or and/or extrapolation, in order to determine the value of Cn at Tᵣₑᵥ, shown in FIG. 6. The latter approach will allow the method to be carried out in a shorter time span.

The next step is to reverse the flow at Tᵣₑᵥ (cf. FIGS. 5 and 6) as described above, i.e. a blood flow in a second direction is created in which the venous line 6 carries treated blood from the dialyzer 11 via arterial needle 5a to the upstream position of the blood access. The arterial line 5 draws in blood from the downstream position via venous needle 6a towards the dialyzer 11.

The effect of this measure is a further increase in the effluent conductivity, which after the flow reversal is referred to as Cr. Cr will reach a semi stable value only asymptotically. The value of Cr may be determined after it has become substantially stable, as shown in FIG. 5. The value of Cr may be extrapolated backwards to the point in time of the flow reversal Tᵣₑᵥ. Alternatively, the value may be determined while the conductivity is still increasing by estimating which substantially stable value Cr would have reached at Tᵣₑᵥ after an equilibrium has been established by using numerical methods such as curve fitting or extrapolation, as shown in FIG. 6.

The volumes in the dialyzer and connecting tubes that need to be exchanged cause the delay. During the delay period, changes in other parameters may occur and could influence the measurement negatively. The preferred method uses therefore the values extrapolated, to the point in time where the flow reversal took place. The above techniques allow estimating the value of Cn and of Cr at the same time Tr, thereby increasing the accuracy in Qa calculation.

Unit 85 may then calculate the fluid flow rate in the blood access in accordance with the formula:

Qa=(Tr−Quf)*(Cr−Ci)/(Cn−Cr),

wherein:
Qa=fluid flow rate in the blood access
Tr=transport rate of substances through the semipermeable membrane
Ci=dialysis liquid conductivity upstream the treatment unit or dialyzer 11
Cn=effluent conductivity referring to the dialysis liquid before flow reversal
Cr=effluent conductivity referring to the dialysis liquid after flow reversal
Quf=ultrafiltration flow rate (Quf).

The transport rate may be based on experience values of a particular dialyzer, such as the clearance, calculated from dialyzer capacity and flow rates or measured by comparing a pre-dialysis blood sample with an initial dialysis liquid urea concentration. Alternatively the transport rate (Tr) corresponds to measured effective ionic dialysance (D) or to measured clearance K of the dialyzer, preferably the urea clearance value. The ultrafiltration flow rate Quf is on conventional dialysis machines continuously measured and monitored. The equation can therefore be solved and the fluid flow rate in the blood access is determined. Alternatively to what described above with reference to FIGS. 5,6, the measurement of Qa may be obtained by first configuring the lines in the reversed configuration. Then a change in conductivity or concentration (for instance by means of a step increase or decrease in the concentration of defined solutes in the dialysis liquid) is created and finally the concentration or conductivity of the dialysis liquid downstream the dialyzer is measured both for the liquid in reversed condition and for the liquid in normal condition. This second approach is convenient if the Qa measurement is carried out at the beginning of the dialysis session. Indeed the patient can be first connected to the treatment apparatus with the lines in reversed configuration; then when necessary the lines are reversed, the Qa calculated and the treatment can prosecute normally at high efficiency with no need of further line switching as the line are already in normal configuration.

In case the method is performed starting from the reversed configuration, then the Qa is still calculated as a function of the above-identified parameters.

If Tr is determined from the measured clearance K or the measured effective ionic dialysance D in vivo values obtained when said venous and arterial lines are in the normal configuration, the fluid flow rate (Qa) in said blood access is calculated by the formula Qa=(Tr−Quf)*(Cr−Ci)/(Cn−Cr), where Tr is the transport rate when the lines are in the normal configuration.

If Tr is obtained from the measured clearance K or the measured effective ionic dialysance D in vivo values obtained when said venous and arterial lines are in the reversed configuration, the fluid flow rate (Qa) in said blood access is calculated by the formula Qa=(Trᵣ−Quf)*(Cᵣ−Ci)/(Cₙ−Cᵣ)+Trᵣ, where Trᵣ is the transport rate when the lines are in the reversed configuration.

The measured clearance K or the measured effective ionic dialysance D in vivo values can obtained by the following steps:
a. passing a third dialysis liquid through the second chamber of said treatment unit, said dialysis liquid presenting a concentration for at least one substance, then
b. obtaining a third post treatment unit conductivity of the dialysis liquid or third post treatment unit concentration of said substance for the third dialysis liquid,
c. at least for a second time interval, increasing or decreasing the concentration of the substance in the third dialysis liquid for passing a fourth liquid through the second chamber inlet, said fourth liquid having a concentration of at least said substance different from the concentration of the same substance in the third liquid,
d. obtaining a fourth post treatment unit conductivity of the dialysis liquid or fourth post treatment unit concentration of said substance for the fourth dialysis liquid,
   calculating the in vivo value of K or D as a function of said third post treatment unit concentration or conductivity and of said fourth post treatment unit concentration or conductivity.

In particular the measured clearance K or the measured ionic dialysance D can be determined during the time interval T so as to use the change in conductivity necessary for the implementation of the present invention. In this case a separate modification of the liquid arriving at the second chamber 13 is not necessary and the third liquid corresponds to the first liquid (before the step in FIGS. 5,6) and the fourth liquid corresponds to the second liquid (after the step in FIGS. 5,6).

Practically if only ions concentration is altered, and again referring to the example of FIG. 5,

$$Tr = K$$

$$K = ( D + U ) \cdot ( 1 - \frac{Δ {C}_{o}}{Δ {C}_{i}} )$$

$$\frac{Δ {C}_{o}}{Δ {C}_{i}}$$

being the inverse of the rate between the step in conductivity of the dialysis fluid at the dialyser inlet and the corresponding step of the dialysis liquid at the outlet of the dialyzer

$$A = ( K - U ) \cdot ( \frac{{C}_{i} - {C}_{r}}{{C}_{r} - {C}_{n}} )$$

According to another feature of the invention a method and corresponding apparatus is provided for checking if the arterial and venous lines are in said normal or in said reversed configuration is provided for. This check can be executed at any time during treatment. If the check is carried out after the lines switching it can serve to provide an alert signal in case the operator (manual switching) or the apparatus (automatic switching) failed to return the lines in the normal configuration.

The step of checking if the arterial and venous lines are in the normal or in the reversed configuration comprises the following steps:

Determining the in vivo value of a parameter selected in the group comprising:
a. Effective ionic dialysance D or
b. Effective clearance K or
c. a parameter proportional to effective ionic dialysance or
d. a parameter proportional to effective clearance,

Comparing the in vivo value of said parameter with a corresponding threshold value for determining if the venous and arterial lines are in said normal or in said reversed configuration.

In case effective ionic dialysance D is used, any known method for in vivo determination of D can be used, such as the one described in EP 658 352, which is herein incorporated by reference.

A simple way of determining D comprises the steps of:
passing a third dialysis liquid through the second chamber inlet of said treatment unit, said dialysis liquid presenting a concentration for at least one substance, then
obtaining a third post treatment unit conductivity of the dialysis liquid or third post treatment unit concentration of said substance for the third dialysis liquid,
at least for a second time interval, increasing or decreasing the concentration of the substance in the third dialysis liquid for passing a fourth liquid through the second chamber inlet, said fourth liquid having a concentration of at least said substance different from the concentration of the same substance in the third liquid,
obtaining a fourth post treatment unit conductivity of the dialysis liquid or fourth post treatment unit concentration of said substance for the fourth dialysis liquid,
calculating the in vivo value of D as a function of said third post treatment unit concentration or conductivity and of said fourth post treatment unit concentration or conductivity.

Once obtained the effective ionic dialysance value D, than D can be compared with a threshold value, which can be a set value or a calculated value or a measured value.

In vivo determination of D can of course be carried out during the time interval T.

In case the step of checking if the arterial and venous lines are in said normal or in said reversed configuration is carried out during the time interval T, then the following alternative procedure can be used:
Comparing said obtained first post-treatment unit conductivity of the dialysis liquid or first post treatment unit concentration of said substance in the dialysis liquid with said obtained second post treatment unit conductivity of the dialysis liquid or second post treatment unit concentration of said substance in the dialysis liquid,
Determining if said conductivity or concentration are increasing after the switching step. Indeed as can be seen in FIG. 5, if the conductivity of blood I lower than that of the dialysis liquid, after switching into reversed condition, a sudden increase in conductivity of the dialysis liquid downstream the dialyzer is registered.

The upstream conductivity cell should preferably calibrated relative to the downstream conductivity cell 20 for improved accuracy. Preferably temperature compensated conductivity cells are used to improve the accuracy of the method.

The value for Ci may be determined by measuring the conductivity of the dialysis fluid before it enters the dialyzer. Alternatively the set value for the dialysis fluid conductivity may be used, since the actual conductivity will only differ marginally from the set value as dialysis monitors control the conductivity of the dialysis fluid very accurately.

1. A method for determining a fluid flow rate (Qa) in a blood access having an upstream position and a downstream position using a blood treatment apparatus, the blood treatment apparatus including:
a blood treatment unit having a semi permeable membrane separating a first chamber through which blood removed from said blood access passes from a second chamber through which dialysis liquid passes,
an arterial line connected to an inlet of the first chamber, and
a venous line connected to an outlet of the first chamber,
said arterial and venous lines configured to provide:
a normal configuration in which said arterial line carries blood from said upstream position of said blood access and said venous line carries blood towards said downstream position of said blood access, and
a reversed configuration in which said arterial line carries blood from said downstream position of said blood access and said venous line carries blood towards said upstream portion of said blood access,
said method comprising:
passing a first dialysis liquid into the second chamber of the treatment unit through a second chamber inlet, said first dialysis liquid presenting a treatment concentration for at least one substance,
increasing or decreasing at a time (Ti) the concentration of the at least one substance in the first dialysis liquid to provide a second dialysis liquid,
passing the second dialysis liquid into the second chamber of the treatment unit through the second chamber inlet during a time interval (T), wherein the second dialysis liquid has a concentration of the at least one substance or a conductivity (Ci) that is kept substantially constant and different from that present in blood,
switching the venous and arterial lines during said time interval (T) between one of said normal and reversed configurations to the other of said normal and reversed configurations,
obtaining, from the second dialysis liquid leaving the second chamber of the treatment unit, a first post treatment unit conductivity of the second dialysis liquid or a first post treatment unit concentration of said substance (Cn; Cr) in the second dialysis liquid, said first post treatment unit conductivity or first post treatment unit concentration (Cn; Cr) obtained when the venous and arterial lines configured according to one of said normal or reversed configuration before switching the venous and arterial lines and during said time interval (T),
obtaining a second post treatment unit conductivity of the second dialysis liquid or post treatment unit concentration of said substance (Cr; Cn) in the second dialysis liquid, said second post treatment unit conductivity or second post treatment unit concentration (Cr; Cn) obtained when the venous and arterial lines are configured according to the other of said normal or reversed configuration after switching of the venous and arterial lines during said time interval (T);
determining the transport rate (Tr; Trᵣ) of ions though the semi permeable membrane,
calculating the fluid flow rate (Qa) in said blood access as a function of values of:
said transport rate (Tr; Trᵣ),
said first post treatment unit conductivity or first post treatment unit concentration (Cn; Cr),
said second post treatment unit conductivity or second post treatment unit concentration (Cr; Cn), and
the concentration of the at least one substance in the second dialysis liquid passing through the second chamber inlet or the conductivity (Ci) of the second dialysis liquid passing through the second chamber inlet.

2. Method according to claim 1, wherein said at least one substance comprises one or more ions.

3. Method according to claim 1, wherein, during said time interval (T):
a. said arterial and venous lines are configured according to the normal configuration for obtaining said first concentration or first conductivity (Cn),
b. the arterial and venous lines are configured from the normal configuration to the reversed configuration for obtaining said second concentration or conductivity (Cr), and
c. the arterial and venous lines are returned from the reversed configuration to the normal configuration for starting of a blood treatment.

4. Method according to claim 1, wherein, during said time interval (T):
a. said arterial and venous lines are configured according to the reversed configuration for obtaining said first post treatment unit concentration or conductivity (Cr), and
b. the arterial and venous lines are configured from the reversed configuration to the normal configuration for obtaining said second concentration or conductivity (Cn).

5. Method according to claim 1, wherein the method comprises checking if the arterial and venous lines are in said normal configuration or in said reversed configuration.

6. Method according to claim 5, wherein checking if the arterial and venous lines are in the normal configuration or in the reversed configuration comprises:
a. determining an in vivo value of a parameter selected from the group comprising:
i. effective ionic dialysance (D),
ii. effective clearance (K),
iii. a parameter proportional to effective ionic dialysance,
iv. a parameter proportional to effective clearance,
b. comparing the in vivo value of said parameter with a corresponding threshold value for determining if the venous and arterial lines are in said normal configuration or in said reversed configuration.

7. Method according to claim 6, wherein determining the in vivo value of said parameter comprises:
a. passing a third dialysis liquid into the second chamber of the treatment unit through the second chamber inlet, said third dialysis liquid presenting a concentration for at least one substance,
b. obtaining a third post treatment unit conductivity of the third dialysis liquid or third post treatment unit concentration of said at least one substance for the third dialysis liquid,
c. increasing or decreasing the concentration of the at least one substance in the third dialysis liquid for a second time interval to provide a fourth dialysis liquid,
d. passing the fourth dialysis liquid into the second chamber of the treatment unit through the second chamber inlet, said fourth dialysis liquid having a concentration of the at least one substance different from the concentration of the same substance in the third dialysis liquid,
e. obtaining a fourth post treatment unit conductivity of the fourth dialysis liquid after the fourth dialysis liquid leaves the second chamber or fourth post treatment unit concentration of said substance for the fourth dialysis liquid after the fourth dialysis liquid leaves the second chamber,
f. calculating the in vivo value of said parameter as a function of said third post treatment unit concentration or third post treatment unit conductivity and of said fourth post treatment unit concentration or fourth post treatment unit conductivity.

8. Method according to claim 6, wherein determining the in vivo value of said parameter is carried out during the time interval (T).

9. Method according to claim 5, wherein checking if the arterial and venous lines are in said normal configuration or in said reversed configuration is carried out during said first time interval (T).

10. Method according to claim 5, wherein checking if the arterial and venous lines are in said normal configuration or in said reversed configuration comprises:
comparing said obtained first post-treatment unit conductivity of the second dialysis liquid with said obtained second post treatment unit conductivity of the second dialysis liquid or comparing said obtained first post treatment unit concentration of said at least one substance in the second dialysis liquid with said second post treatment unit concentration of said at least one substance in the second dialysis liquid,
determining if said conductivity or said concentration are increasing after switching the venous and the arterial lines during said time interval (T).

11. Method according to claim 1, further comprising obtaining the ultrafiltration flow rate (Quf).

12. Method according to claim 11, wherein the transport rate of ions though the semi permeable membrane (Tr) is determined from the measured clearance (K) or the measured effective ionic dialysance (D) in vivo values obtained when said venous and arterial lines are in the normal configuration, the fluid flow rate (Qa) in said blood access being calculated by the formula Qa=(Tr−Quf)*(Cr−Ci)/(Cn−Cr), where (Tr) is the transport rate when the venous and arterial lines are in the normal configuration.

13. Method according to claim 11, wherein the transport rate of ions though the semi permeable membrane (Trᵣ) is determined from the measured clearance (K) or the measured effective ionic dialysance (D) in vivo values obtained when said venous and arterial lines are in the reversed configuration, the fluid flow rate (Qa) in said blood access being calculated by the formula Qa=(Trᵣ−Quf)*(Cr−Ci)/(Cn−Cr)+Trᵣ.

14. Method according to claim 12, wherein the measured clearance (K) or the measured ionic dialysance (D) are in vivo values determined during the time interval (T).

15. Method according to claim 13, wherein the measured clearance (K) or the measured ionic dialysance (D) are in vivo values determined during the time interval (T).

16. Method according to claim 1 comprising:
a. changing the conductivity in the first dialysis liquid upstream from the treatment unit to provide the second dialysis liquid,
b. keeping the conductivity of the second dialysis liquid upstream from the treatment unit substantially constant during said time interval (T),
c. waiting a delay after starting to change the conductivity of the first dialysis liquid and then determine the time (T0) when a prefixed change in conductivity occurs in the second dialysis liquid leaving the second chamber of the treatment unit,
d. measuring a plurality of first values of conductivity of the second dialysis liquid leaving the second chamber of the treatment unit after said time (T0),
e. calculating the first post-treatment unit conductivity of said second dialysis liquid from said plurality of values;
f. switching the venous and arterial lines from one of said normal and reversed configurations to the other of said normal and reversed configurations;
g. measuring a plurality of second values of conductivity of the second dialysis liquid leaving the second chamber of the treatment unit after switching the venous and arterial lines from one of said normal and reversed configurations to the other of said normal and reversed configurations,
h. calculating the second post-treatment unit conductivity of said second dialysis liquid leaving the second chamber of the treatment unit from said plurality of values.

17. Method according to claim 16, wherein the switching of the venous and arterial lines from one of said normal and reversed configurations to the other of said normal and reversed configurations occurs at time (Trev), and wherein the plurality of second values of conductivity of the second dialysis liquid are measured after a delay from time (Trev).

18. Method according to claim 17, wherein the plurality of second values of the conductivity are obtained continuously or intermittently and the second post-treatment unit conductivity (Cr) of the second dialysis liquid leaving the second chamber of the treatment unit at the time (Trev) is determined by extrapolating the plurality of second values backwards to the time of the switching (Trev).

19. Method for checking the operating configuration of the arterial and venous lines of a blood treatment apparatus, the blood treatment apparatus including:
a blood treatment unit having a semi permeable membrane separating a first chamber through which blood removed from said blood access passes from a second chamber through which dialysis liquid passes,
an arterial line connected to an inlet of the first chamber, and
a venous line connected to an outlet of the first chamber,
said arterial and venous lines configured to provide at least a normal configuration in which said arterial line carries blood from an upstream position of a blood access, and said venous line carries blood towards a downstream position of said blood access, and
a reversed configuration in which said arterial line carries blood from said downstream position of said blood access and said venous line carries blood towards said upstream portion of said blood access, said method comprising:
passing a dialysis liquid through the second chamber of said treatment unit at least for a time interval (T), said dialysis liquid having a concentration (Ci) of at least one substance different from the concentration of the same substance in blood upstream from the treatment unit,
determining the in vivo value of a parameter selected from the group comprising:
effective ionic dialysance (D),
effective clearance (K),
a parameter proportional to effective ionic dialysance, or
a parameter proportional to effective clearance,
comparing the in vivo value of said parameter with a corresponding threshold value which is one of a set value or a calculated value,
determining if the venous and arterial lines are in said normal configuration or in said reversed configuration depending upon the outcome of said comparing.

20. A method for determining a fluid flow rate (Qa) in a blood access having an upstream position and a downstream position using a blood treatment apparatus, the blood treatment apparatus including:
a blood treatment unit having a semi permeable membrane separating a first chamber through which blood removed from said blood access passes from a second chamber through which dialysis liquid passes,
an arterial line connected to an inlet of the first chamber, and
a venous line connected to an outlet of the first chamber,
said arterial and venous lines configured to provide:
at least a normal configuration in which said arterial line carries blood from said upstream position of said blood access and said venous line carries blood towards said downstream position of said blood access, and
at least a reversed configuration in which said arterial line carries blood from said downstream position of said blood access and said venous line carries blood towards said upstream portion of said blood access,
said method comprising:
configuring the arterial and venous lines in the reversed configuration,
increasing or decreasing at a time (Ti) a concentration of at least one substance in dialysis liquid delivered into the second chamber of the treatment unit through a second chamber inlet which—during a time interval (T)—has a concentration of the at least one substance or a conductivity (Ci) that is kept substantially constant and different from that present in blood, then
obtaining, downstream from the treatment unit, a first post treatment unit conductivity of the dialysis liquid leaving the second chamber of the treatment unit or a first post treatment unit concentration of said substance (Cn; Cr) in the dialysis liquid leaving the second chamber of the treatment unit, said first post treatment unit conductivity or first post treatment unit concentration (Cn; Cr) obtained when the venous and arterial lines are configured according to said reversed configuration, then
switching the venous and arterial lines, during said time interval (T), from said reversed configuration to the normal configuration, then
obtaining, downstream from the treatment unit, a second post treatment unit conductivity of the dialysis liquid leaving the second chamber of the treatment unit or a second post treatment unit concentration of said substance (Cr; Cn) in the dialysis liquid leaving the second chamber of the treatment unit, said second post treatment unit conductivity or second post treatment unit concentration (Cr; Cn) obtained when the venous and arterial lines are configured according to said normal configuration,
determining the transport rate (Tr; Trᵣ) of ions though the semi permeable membrane,
calculating the fluid flow rate (Qa) in said blood access as a function of values of:
said transport rate (Tr; Trᵣ),
said first post treatment unit conductivity or first post treatment unit concentration (Cn; Cr),
said second post treatment unit conductivity or second post treatment unit concentration (Cr; Cn), and
the concentration of the at least one substance or the conductivity (Ci) in the dialysis liquid delivered into the second chamber of the treatment unit through the second chamber inlet,
continue passing blood through the first chamber of the treatment unit and dialysis liquid through the second chamber of the treatment unit without switching the arterial and venous lines out of the normal configuration.

21. The method of claim 20 further comprising evaluating the configuration of said arterial and venous lines by determining if said second post treatment unit conductivity or second post treatment concentration are increasing or decreasing after switching the arterial and venous lines from the reversed configuration to the normal configuration.

22. A method for determining a fluid flow rate (Qa) in a blood access having an upstream position and a downstream position using a blood treatment apparatus, the blood treatment apparatus including:
a blood treatment unit having a semi permeable membrane separating a first chamber through which blood removed from said blood access passes from a second chamber through which dialysis liquid passes,
an arterial line connected to an inlet of the first chamber and a venous line connected to an outlet of the first chamber, said arterial and venous lines configured to provide: at least a normal configuration in which said arterial line carries blood from said upstream position of said blood access and said venous line carries blood towards said downstream position of said blood access, and at least a reversed configuration in which said arterial line carries blood from said downstream position of said blood access and said venous line carries blood towards said upstream portion of said blood access,
a valve configured to connect the arterial line with the upstream position of the blood access and the venous line with the downstream position of the blood access in a first position of said valve, said valve further configured to connect the arterial line with the downstream position of the blood access and the venous line with the upstream position of the blood access when the valve is in a second position,
said method comprising:
passing a first dialysis liquid into the second chamber of the treatment unit through a second chamber inlet, said first dialysis liquid presenting a treatment concentration for at least one substance,
increasing or decreasing at a time (Ti) the concentration of the at least one substance in the first dialysis liquid to provide a second dialysis liquid,
passing the second dialysis liquid into the second chamber through the second chamber inlet which—during a time interval (T)—has a concentration of the at least one substance or a conductivity (Ci) that is kept substantially constant and different from that present in blood,
operating a valve to switch the venous and arterial lines, during said time interval (T), between one of said normal and reversed configurations to the other of said normal and reversed configurations,
obtaining, downstream from the treatment unit, a first post treatment unit conductivity of the second dialysis liquid leaving the second chamber of the treatment unit or a first post treatment unit concentration of said substance (Cn; Cr) in the second dialysis liquid leaving the second chamber of the treatment unit, said first post treatment unit conductivity or first post treatment unit concentration (Cn; Cr) obtained before switching the venous and arterial lines between said normal and reversed configurations during said time interval T,
obtaining, downstream from the treatment unit, a second post treatment unit conductivity of the second dialysis liquid leaving the treatment unit or second post treatment unit concentration of said substance (Cr; Cn) in the second dialysis liquid leaving the treatment unit, said second post treatment unit conductivity or second post treatment unit concentration (Cr; Cn) obtained before switching the venous and arterial lines between said normal and reversed configurations during said time interval (T),
determining the transport rate (Tr; Trᵣ) of ions though the semi permeable membrane,
calculating the fluid flow rate (Qa) in said blood access as a function of values of:
said transport rate (Tr; Trᵣ),
said first post treatment unit conductivity or first post treatment unit concentration (Cn; Cr),
said second post treatment unit conductivity or second post treatment unit concentration (Cr; Cn), and
the concentration of the at least one substance or the conductivity (Ci) in the second dialysis liquid delivered to the second chamber of the treatment unit through the second chamber inlet.

23. The method of claim 22 comprising checking if the arterial and venous lines are in said normal configuration or in said reversed configuration after determining the fluid flow rate (Qa) in the blood access and
providing an alert signal if the arterial and venous lines are not in the normal configuration after determining the fluid flow rate (Qa) in the blood access.
